(19) United States
(12) Patent Application Publication
(10) Pub. No.: US 2013/0166039 A1
(43) Pub. Date: Jun. 27, 2013

(54) OSSEOUS IMPLANT AND METHODS OF ITS MAKING AND USE

(71) Applicant: Micropen Technologies Corporation, Honeoye Falls, NY (US)

(72) Inventor: Lori J. Shaw-Klein, Rochester, NY (US)

(73) Assignee: MICROPEN TECHNOLOGIES CORPORATION, Honeoye Falls, NY (US)

(21) Appl. No.: 13/719,424

(22) Filed: Dec. 19, 2012

Related U.S. Application Data
(60) Provisional application No. 61/578,671, filed on Dec. 21, 2011.

(51) Int. Cl. A61F 2/28

(52) U.S. Cl. CPC A61F 2/28
USPC 623/23.49; 427/2.26

(57) ABSTRACT

The present invention relates to an osseous implant for osteogenesis promotion and maintenance, the implant having an exposed surface, and the improvement comprising an electrical circuit attached to the osseous implant. At least a portion of the electrical circuit comprises a trace of conductive particles deposited on the exposed surface of the osseous implant. The present invention also relates to a method of promoting and maintaining osteogenesis by implanting the osseous implant into a subject. Current is passed through the electrical circuit under conditions effective to promote and maintain osteogenesis in the subject. Also disclosed is a method of making an osseous implant for osteogenesis promotion and maintenance.

This application claims the benefit of U.S. Provisional Patent Application Ser. No. 61/578,671, filed Dec. 21, 2011, which is hereby incorporated by reference in its entirety.

### FIELD OF THE INVENTION

The present invention relates to an osseous implant and methods of its making and use.

### BACKGROUND OF THE INVENTION

Implants, prostheses, and scaffolds are often used to join or replace damaged bone or cartilage. Examples include orthopedic implants, such as femoral and acetabular implants, used for hip replacements; knee joint replacements; screws and fracture plates intended to hold bones in place during healing; cochlear or dental implants; spinal implants, including pedicle screws; and open scaffold materials designed to promote tissue growth.

There is particular interest in accelerating and enhancing the growth of bone, known as osteogenesis and osseointegration, in and around such implants to encourage fixation so that the implants are mechanically anchored and more readily accepted by the body. Many approaches are followed to enhance tissue growth into such surfaces, including surface modification with chemical species which resemble or duplicate components of the cartilage or bone itself. Another surface modification approach involves roughening or introducing a layer with open porosity that provides a geometry and surface area optimized for ingrowth of the desired tissue. Yet another approach to enhancing osseointegration is electrical stimulation in the affected area.

There are three common types of electrical stimulation, each of which functions by up-regulating osteoinductive growth factors, tissue growth factors, or morphogenic proteins depending on the type of stimulation applied. Other small changes in local pH and oxygen levels may also play a role in encouraging bone or tissue growth. In one type of electrical stimulation, a pulsed electromagnetic field is applied externally, generating a small electrical current in the desired area. In another type of electrical stimulation, known as capacitive coupling, a low-voltage alternating current is applied externally over the fracture or fusion site. In a final type of electrical stimulation, a direct current may be applied through an implanted electrode.

PCT Publication No. WO 2004/066851 to Madjar et al. describes electrodes optimized for endosseous implants. An inlaid electrode is described such that the external surface topography of the implant is unaffected. Inlaid electrodes are formed by sinking the conductive material in a channel or impression which must first be provided on the surface of the implant, also known as a “damascene” conductor.

U.S. Patent Application Publication No. 2007/0179562 to Nycz describes an implantable tissue growth stimulator, particularly suitable for an acetabular cup in a hip prosthesis. The electrodes are disposed in a sheath which is situated between the bone and the conductor. This malleable sheath may potentially be altered and positioned by the surgeon during implantation to achieve the most optimal position for stimulation.

U.S. Pat. No. 7,172,594 to Biscup describes a screw, nail, or post designed for implantation which encourages bone growth through electrical stimulation. Electrodes are described which may be located on a screw, for example, and connected through a channel to a power source. Such connections are typically wires.

U.S. Patent Application Publication No. 2010/0152864 to Isaacson et al. describes a non-invasive electrical stimulation system designed to improve bone fixation for amputees already having a metallic implant. This metallic implant can act as one electrode, while the other is provided externally. While this approach provides great simplicity, it does not provide a method by which very specific areas of the implant may be targeted for electrical stimulation.

The present invention is directed to overcoming these and other deficiencies in the art.

### SUMMARY OF THE INVENTION

One aspect of the present invention relates to an osseous implant for osteogenesis promotion and maintenance, the implant having an exposed surface, and the improvement comprising an electrical circuit attached to the osseous implant. At least a portion of the electrical circuit comprises a trace of conductive particles deposited on the exposed surface of the osseous implant.

Another aspect of the present invention relates to a method of promoting and maintaining osteogenesis. This method involves providing the osseous implant of the present invention and implanting the osseous implant into a subject. Current is passed through the electrical circuit under conditions effective to promote and maintain osteogenesis in the subject.

A further aspect of the present invention relates to a method of making an osseous implant for osteogenesis promotion and maintenance. This method involves providing an osseous implant with an exposed surface; applying a conductive ink composition comprising conductive particles in a solvent on a surface of the osseous implant to form an electrical circuit; and curing the conductive ink composition under conditions effective to form an electrical circuit comprising a trace of conductive particles deposited on the exposed surface of the osseous implant.

The present invention relates to direct current electrical stimulation through an implanted electrode, specifically, an implanted electrode designed for implants intended for skeletal applications in which osseointegration or osteogeneration is desired. Such electrodes are required for direct current stimulation of tissue in-growth. The present invention relates to such electrodes, leads connected to those electrodes, and insulating layers as required, deposited directly on the implant surface by direct write methods, thereby providing a means of specifically and precisely placing such an electrode, and strongly affixing the electrode to the surface. This approach also allows for a smoother, more conformal introduction of electronics onto the surface of the implant, leading to less disruption and discomfort as the implant is introduced.

The present invention is an improvement over PCT Publication No. WO 2004/066851 to Madjar et al., which requires extra process steps to provide in-laid electrodes compared with the simplicity of direct writing techniques used in the present invention. The present invention obviates the need for a carrier sheath, as required by U.S. Patent Application Publication No. 2007/0179562 to Nycz, because the electrodes of the present invention are written directly on the implant surface. Moreover, the present invention is an improvement over U.S. Pat. No. 7,172,594 to Biscup, because the difficulty of providing the channels, wires, and electrodes is avoided or simplified by directly depositing the necessary features through directly writing them on the surface of the screw, nail, or post. Furthermore, the present invention excels over U.S. Patent Application Publication No. 2010/0152864 to Isaacson et al. in flexibility for directly applying electrodes on precisely those areas of an implant most appropriate and responsive to osseointegrative electrical stimulation.

### BRIEF DESCRIPTION OF THE DRAWINGS

FIGS. 1A-C are schematic illustrations of three different views of a femoral stem of a hip implant. FIG. 1A and FIG. 1C are opposite side views of the femoral stem of the hip implant. FIG. 1B is a front view of the femoral stem of the hip implant. As shown in the side view of FIG. 1C, the implant includes an insulating substrate layer, an electrical stimulation electrode, and an insulating top layer formed on an exposed surface of the implant where bone growth is not desired. A lead electrically connects the electrical stimulation electrode to an external current source.

FIGS. 2A-C are horizontal, cross-sectional views showing a sequence of steps, from FIG. 2A to FIG. 2C, of the formation of the electrical stimulation electrode on the exposed surface of the hip implant of FIG. 1, according to one embodiment of the present invention. FIG. 2C is a cross-sectional view of the hip implant of FIG. 1 at a region without an insulating top layer.

FIGS. 3A-D are horizontal, cross-sectional views showing a sequence of steps, from FIG. 3A to FIG. 3D, of the formation of the electrical stimulation electrode on the exposed surface of the implant of FIG. 1, according to one embodiment of the present invention. FIG. 3D is a cross-sectional view of the hip implant of FIG. 1 at a region with an insulating top layer.

FIG. 4 is a front view of a schematic illustration of a dental implant having an electrical circuit attached to the implant by direct writing to form a trace of conductive particles deposited on an exposed surface of the osseous implant according to one embodiment of the present invention. Bone into which the implant is embedded is cut away to show features of the implant. The implant has on its exposed surface an insulating substrate layer, electrical stimulation electrode, lead, and insulating top layer. The electrode is formed on an exposed surface of the implant in an area in which bone growth is desired.

FIGS. 5A-C are horizontal, cross-sectional views showing a sequence of steps, from FIG. 5A to FIG. 5C, of the formation of the electrical stimulation electrode on the exposed surface of the dental implant of FIG. 4, according to one embodiment of the present invention. FIG. 5C is a cross-sectional view of the dental implant of FIG. 4 at a region without an insulating top layer.

FIGS. 6A-D are vertical, cross-sectional views showing a sequence of steps, from FIG. 6A to FIG. 6D, of the formation of the electrical stimulation electrode on the exposed surface of the dental implant of FIG. 4, according to one embodiment of the present invention. FIG. 6D is a cross-sectional view of the dental implant of FIG. 4 at a region with an insulating top layer.

FIG. 7 is a schematic illustration of a front view of a bone scaffold segment with an electrical stimulation electrode, lead, and insulating top layer applied to the scaffold surface in an area in which bone growth is desired, according to one embodiment of the present invention.

FIGS. 8A-B are horizontal, cross-sectional views showing a sequence of steps, from FIG. 8A to FIG. 8B, of the formation of the electrical stimulation electrode on the exposed surface of the bone scaffold segment of FIG. 7, according to one embodiment of the present invention. FIG. 8B is a cross-sectional view of the bone scaffold segment of FIG. 7 at a region without an insulating top layer.

FIGS. 9A-C are horizontal, cross-sectional views showing a sequence of steps, from FIG. 9A to FIG. 9C, of the formation of the electrical stimulation electrode on the exposed surface of the bone scaffold segment of FIG. 7, according to one embodiment of the present invention. FIG. 9C is a cross-sectional view of the bone segment of FIG. 7 at a region with an insulating top layer.

### DETAILED DESCRIPTION OF THE INVENTION

A first aspect of the present invention relates to an osseous implant for osteogenesis promotion and maintenance, the implant having an exposed surface, and the improvement comprising an electrical circuit attached to the osseous implant. At least a portion of the electrical circuit comprises a trace of conductive particles deposited on the exposed surface of the osseous implant.

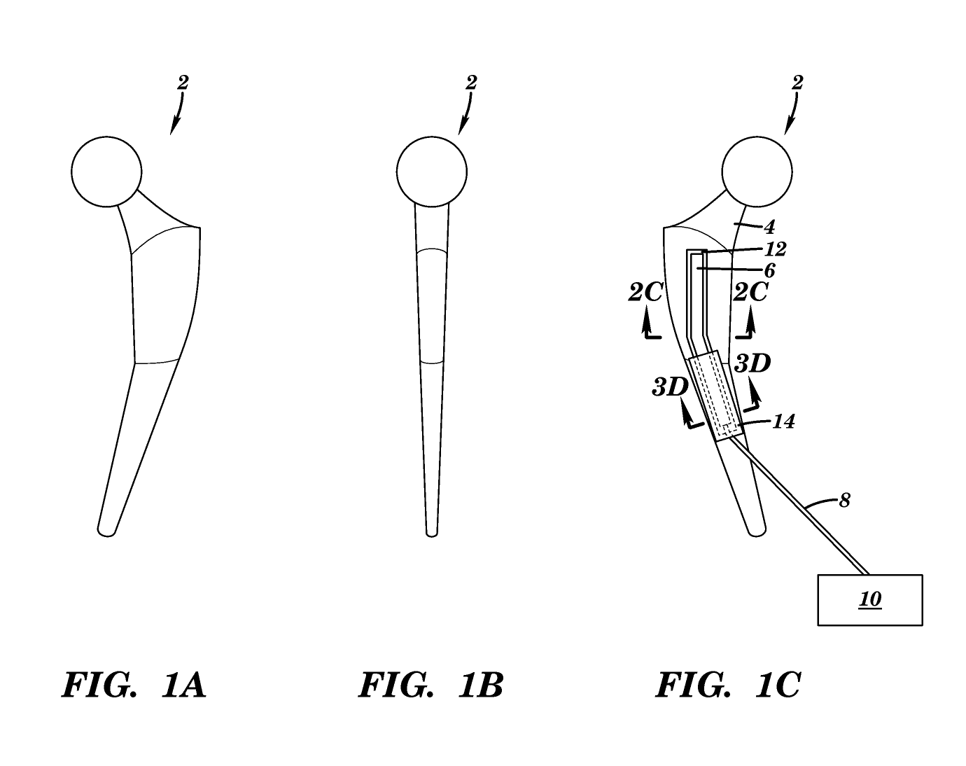

With reference to FIGS. 1A-C, a frontal view (FIG. 1B) and two opposing side views (FIGS. 1A and 1C) of an osseous implant according to one embodiment of the present invention are shown. Osseous implant 2 is a femoral stem of a hip implant. Referring now specifically to FIG. 1C, hip implant 2 has exposed surface 4, to which an electrical circuit is attached. By “attached,” it is meant that at least a portion of the electrical circuit is in physical contact with exposed surface 4. In one embodiment described in more detail infra, the attached electrical circuit is written directly on exposed surface 4 or written directly on an insulating substrate layer on exposed surface 4.

The electrical circuit includes electrical stimulation electrode 6, lead 8, and external current source 10. Lead 8 provides an electrical connection between external current source 10 and electrical stimulation electrode 6. Hip implant 2 also has insulating substrate layer 12 disposed beneath stimulation electrode 6 and positioned between electrical stimulation electrode 6 and exposed surface 4, and insulating top layer 14 formed over a portion of electrical stimulation electrode 6 (and over a portion of insulating substrate layer 12).

Surface 4 of hip implant 2 is an exposed surface of the implant, i.e., an external surface of implant 2 such that, when implanted into a subject, surface 4 is in contact with or is proximal to an area where bone or tissue growth is desired. Thus, particularly suited exposed surfaces of an osseous implant for attachment of an electrical circuit according to the present invention are those surfaces on the implant that may be in contact with or are proximal to bone or tissue where osteogenesis promotion and/or maintenance is desired.

As would be appreciated by those of ordinary skill in the art of osseous implants, such surfaces may include materials selected from metal, polymer, ceramic, or combinations of these materials. For example, orthopedic and dental implants are often constructed of metals, such as stainless steel, titanium and its alloys, cobalt-chromium alloys, and the like. They may also be constructed of bioresorbable metals such as magnesium, if it is desired that the implant be non-permanent. Often, exposed surfaces of osseous implants are roughened to promote bone growth and integration at or near the roughened surface. Osseous implants of the present invention have electrical circuits that can be formed on roughened surfaces of an implant.

Certain ceramic materials are also useful in either constructing implants or coating implants to provide a wear resistant surface, a non-reactive surface, or a surface more compatible with bone or tissue. For example, hydroxyapatite coatings are sometimes applied to metal implant surfaces to encourage bone growth. Commercially available glasses and ceramics are also available and are sometimes used to construct all or part of an osseous implant, and are suitable surfaces for attachment of an electrical circuit according to the present invention. For example, Bioglass (Schott), a specific composition of soda-lime glass, provides a surface with a high level of compatibility with bone.

Polymeric materials are commonly used in implants as well, and are suitable for attachment of an electrical circuit according to the present invention. For example, polyethylene may be used in acetabular hip implants; a parylene coating may be provided over metal or ceramic surfaces to inhibit migration of potentially destructive components; and resorbable polymers such polylactic acid, polycaprolactone, alginate, and the like are used to form porous scaffolds which may be implanted to encourage bone ingrowth and repair. The surface may be further altered to provide porosity, chemical modification, drug delivery, or other characteristics useful for the implant.

Femoral hip implants, such as femoral hip implant 2 of FIGS. 1A-C are normally made of metal, which provide appropriate mechanical stability.

When the exposed surface of an osseous implant is constructed of a conductive material, such as metal, it may be desirable for the electrical circuit, or a portion thereof, to be applied to an insulating substrate layer formed on the exposed surface, rather than disposing the electrical circuit, or a portion thereof, directly on the conductive material. Thus, as illustrated in FIG. 1C, femoral hip implant 2, which is constructed of metal, has insulating substrate layer 12 on a portion of exposed surface 4, upon which the entirety of electrical stimulation electrode 6 is formed. In an alternative embodiment, the electrical stimulation electrode is formed either completely or partly on the exposed surface of the implant.

When employed, the insulating substrate layer may be formed of a material chosen for its electrical properties, adhesion to the substrate, and ability to be deposited in desired locations on the substrate. In one embodiment, the particular material forming the insulating substrate layer is biocompatible and/or bioresorbable with other materials. For example, if an insulating substrate layer and an electrical stimulation electrode are intended to be temporarily formed on the surface of an osseous implant, it may be desirable to choose materials that are biocompatible with each other.

According to one embodiment, an insulating substrate layer is formed on an exposed surface of an osseous implant by screen printing of a dielectric ink. This process is particularly suited for attaching an insulating substrate layer to an exposed surface of an osseous implant, because it can be used to form inorganic layers on, e.g., metallic surfaces. Dielectric inks are well known in the art, and are usually comprised of a number of inorganic materials including, without limitation, a glass-forming binder, as well as an organic solvent vehicle, and various additives including dispersants, surfactants, and the like, to optimize liquid properties.

After being applied to a surface of an implant, dielectric inks are generally fired at high temperatures, in excess of 500° C., to remove all traces of organic material and to fuse the inorganic material to form a continuous film. Examples of sources of commercially available dielectric inks include ESL ElectroScience (King of Prussia, Pa.), DuPont Microcircuit Materials (Wilmington, Del.), and Ferro Electronic Material Systems (Mayfield Heights, Ohio).

Other suitable inks can be formulated to function as insulating substrate layers, which include bioactive or biocompatible ceramics or glasses to encourage overall compatibility of layers.

Polymeric materials are also used to form insulating substrate layers on various surfaces including metals, conductive ceramics, carbon or carbon-filled surfaces, conductive polymers, and the like. Such materials are generally comprised of polymeric materials dissolved or dispersed in appropriate liquid carriers. Ceramic materials may also be dispersed in an ink, but since such inks are cured at low temperatures, or are cured via ultraviolet, electron beam, or other energy methods, the polymeric phase forms a continuous binder and the inorganic materials are generally present as a second phase, providing mechanical reinforcement or enhanced dielectric properties.

Examples of polymeric materials that may be used to manufacture inks suitable for use as insulating substrate layers include, without limitation, epoxy, polyacrylate, silicone or natural rubber, polyester, polyethylene napthalate, polypropylene, polycarbonate, polystyrene, polyvinyl fluoride, ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polyphenylsulfone, polytetrafluoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyvinyl chloride, polyphosphazene, or poly(ε-caprolactone). Copolymers or mixtures of polymers may also be used for the purposes of the present invention. Particularly useful commercially available dielectric polymeric inks are available from, for example, Dymax Corporation, MasterBond, and Henkel Loctite.

At least a portion of the electrical circuit attached to the osseous implant according to the present invention comprises a trace of conductive particles deposited on the exposed surface of the osseous implant. In one embodiment, depositing conductive particles on the exposed surface is carried out by using deposition direct writing techniques. These include screen printing, jetting, laser ablation, pressure driven syringe delivery, inkjet or aerosol jet droplet based deposition, laser or ion-beam material transfer, tip based deposition techniques such as dip pen lithography, or flow-based microdispensing. Particularly preferred deposition techniques are those that have the ability to maintain conformality of a deposited conductive composition or ink and offer precision in placement of the conductive composition or ink, as well as flexibility in design and pattern. A direct writing technique that satisfactorily controls and manipulates, for example, a three dimensional, irregular substrate is Micropenning® using a Micropen (Micropen Technologies Corp., Honeoye Falls, N.Y.). This technique is described in Pique et al., Direct-Write Technologies for Rapid Prototyping Applications: Sensors, Electronics, and Integrated Power Sources, Academic Press (2002), which is hereby incorporated by reference in its entirety. According to this embodiment, attachment of the electrical circuit to the osseous implant involves depositing a conductive ink onto the surface of the implant (or, alternatively, onto an insulating substrate layer on the surface of the implant) at the desired location.

Thus, according to one embodiment, and with reference to FIG. 1C, insulating substrate layer 12 is formed onto surface 4 of implant 2 by applying a dielectric ink via screen printing, followed by curing of the dielectric ink. Electrical stimulation electrode 6 is then applied to portions of insulating substrate layer 12 via Micropenning® direct writing of a conductive ink. According to this embodiment, Micropenning® is a particularly preferred methodology, because it can accommodate an extremely wide range of rheological properties and very high solids levels, as well as excellent three dimensional substrate manipulation capabilities.

Preferred conductive ink materials for forming electrical circuits should be capable of dispersion or dissolution in appropriate liquid medium yielding an ink with rheological properties permitting the desired deposition method. Conductive ink compositions which can yield electrodes can comprise conductive particles such as various metals, for example, copper, silver, gold, palladium, platinum, nickel. These ink compositions can also comprise materials such as various forms of conductive carbon (e.g., graphite or carbon black), conductive ceramics (e.g., tin oxide, vanadium pentoxide, doped versions of the tin oxide, or doped versions of vanadium oxide), or conducting polymers (e.g., polypyrrole, polythiophene, or polyaniline). The conductive inks can also include various combinations, mixtures, or copolymers of the above mentioned materials. If the conductor is provided in particulate form, a polymer may be present to bind the conductive particles together and to provide enhanced adhesion to the substrate. A liquid carrier may be present to disperse the components of the ink, and provide interaction with the substrate, enhancing adhesion. Alternatively, a solvent may be present to dissolve the components of the ink. Further additives can include surfactants, thickeners, dispersants, defoamers and the like. Suitable conductive ink compositions include those described in U.S. Patent Application Publication No. 2010/0119789 to Grande, which is hereby incorporated by reference in its entirety.

As will be appreciated by those of ordinary skill in the art, deposition of traces of conductive particles in, e.g., a conductive ink, requires subsequent curing. Curing may involve air drying, heating, UV application, and other methods well known in the art.

With reference to FIG. 1C, lead 8 may be printed directly on exposed implant surfaces or insulating substrate layers. Leads that form part of the electrical circuit are, according to one embodiment, comprised of the same type of conductive ink material used to form electrical stimulation electrode 6. Alternatively, leads that form part of the electrical circuit are constructed of a different type of material than that used to form electrical stimulation electrode 6. In one embodiment, at least a portion of lead 8 comprises a trace of conductive particles deposited on the exposed surface of the osseous implant.

As illustrated in FIG. 1C, lead 8 may extend away from surface 4 of implant 2 and lead to external current source 10. Thus, it may be desirable or necessary for lead 8 to be formed of more than one type and/or form of material. For example, one segment of lead 8 may be written directly on surface 4 of implant 2, and another segment of lead 8 may comprise an insulating wire bonded to the first segment and leading away from the implant (e.g., to outside the body in which the implant is positioned).

Still referring to FIG. 1C, insulating top layer 14 may be provided in areas in which electrical stimulation is not desired, such as over a portion of printed conductive lead 8, or over areas of electrical stimulation electrode 6 to pattern or select very specific regions for electrical stimulation. Insulating top layer 14 may be deposited via direct writing if high levels of precision are desired, or by coating methods known in the art, including but not limited to, dip coating, gravure, curtain, hopper, flexographic, spray coating, and the like, if a more generalized blanket coating is appropriate. Generally, ceramics or polymers are preferred materials for forming insulating top layer 14, as long as they exhibit sufficient biocompatibility, insulation, and mechanical characteristics as needed in a particular application.

External current source 10 may be any suitable device capable of providing DC, AC, or pulsating current, or any combination thereof. Currents provided by the external current source may be pulsed or continuous. In one embodiment, the external current source is provided outside the body of the subject in which the implant has been implanted. In another embodiment, the external current source is placed inside the body of the subject and operated, e.g., by battery power.

In one embodiment, at least a portion of the electrical circuit attached to the osseous implant is above the exposed surface of the osseous implant. In an alternative embodiment, the entire electrical circuit attached to the osseous implant is above the exposed surface of the osseous implant. By being above the exposed surface, it is meant that the implant is not etched, cut-away, or altered to create, e.g., channels, impressions, or traces in which the electrical circuit or portions thereof can be deposited. Rather, conductive compositions are directly applied to an unaltered surface of the implant to be formed on the surface of the implant.

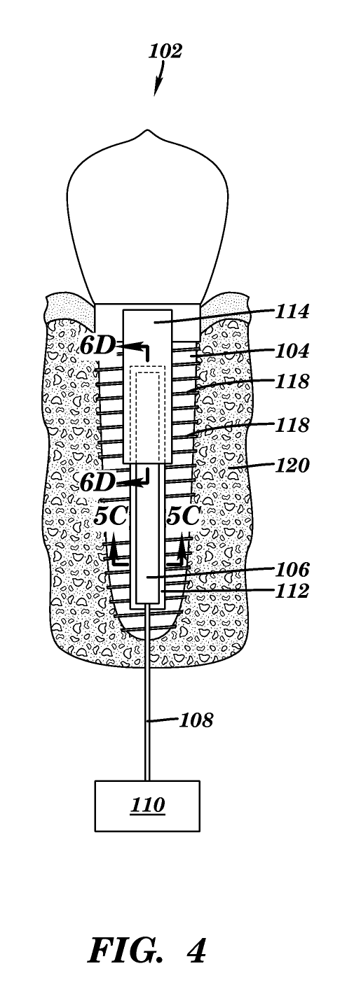

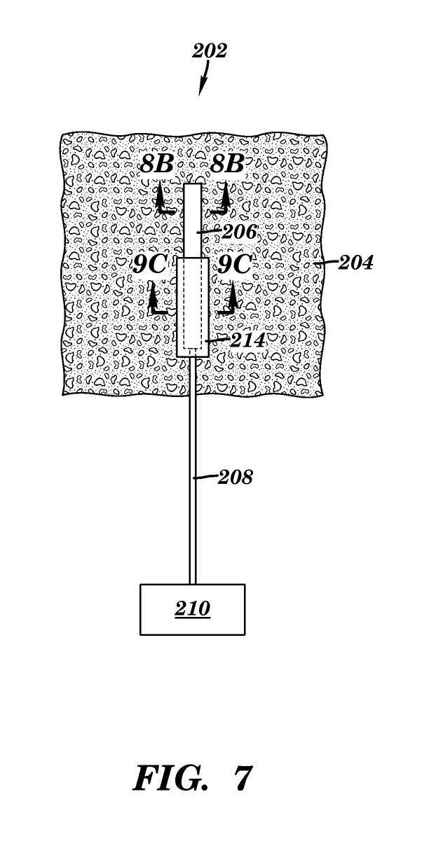

Additional embodiments of osseous implant devices according to the present invention are illustrated in FIG. 4 and FIG. 7, discussed in greater detail infra.

In operation, hip implant 2 is implanted into a subject and external current source 10 sends a current through lead 8 to electrical stimulation electrode 6 to provide electrical stimulation to bone adjacent or proximal surface 4 to promote and maintain osteogenesis at or around the site of implant 2. Insulating top layer 14 is provided to prevent electrical stimulation at areas where insulating top layer 14 covers electrical stimulation electrode 6.

Thus, another aspect of the present invention relates to a method of promoting and maintaining osteogenesis. This method involves providing the osseous implant of the present invention and implanting the osseous implant into a subject. Current is passed through the electrical circuit under conditions effective to promote and maintain osteogenesis in the subject.

By “promoting and maintaining osteogenesis,” it is meant that the method of the present invention is carried out at a site in a subject (e.g., a human or other mammal) where that subject is in need of bone healing. Bone is intended to mean the dense, semi-rigid, porous, calcified connective tissue forming the major portion of the skeleton of most vertebrates, comprising a dense organic matrix and an inorganic, mineral component. Bone is any of numerous anatomically distinct structures making up the skeleton of a vertebrate. The term “osteogenesis” refers to the net development and net formation of bone, including, without limitation the promotion of new bone growth and/or the alleviation of bone resorption. In a particular embodiment, the method of the present invention is carried out to stimulate bone ingrowth into non-cemented prosthetic joints and dental implants.

A further aspect of the present invention relates to a method of making an osseous implant for osteogenesis promotion and maintenance. This method involves providing an osseous implant with an exposed surface; applying a conductive ink composition comprising conductive particles in a solvent on a surface of the osseous implant to form an electrical circuit; and curing the conductive ink composition under conditions effective to form an electrical circuit comprising a trace of conductive particles deposited on the exposed surface of the osseous implant.

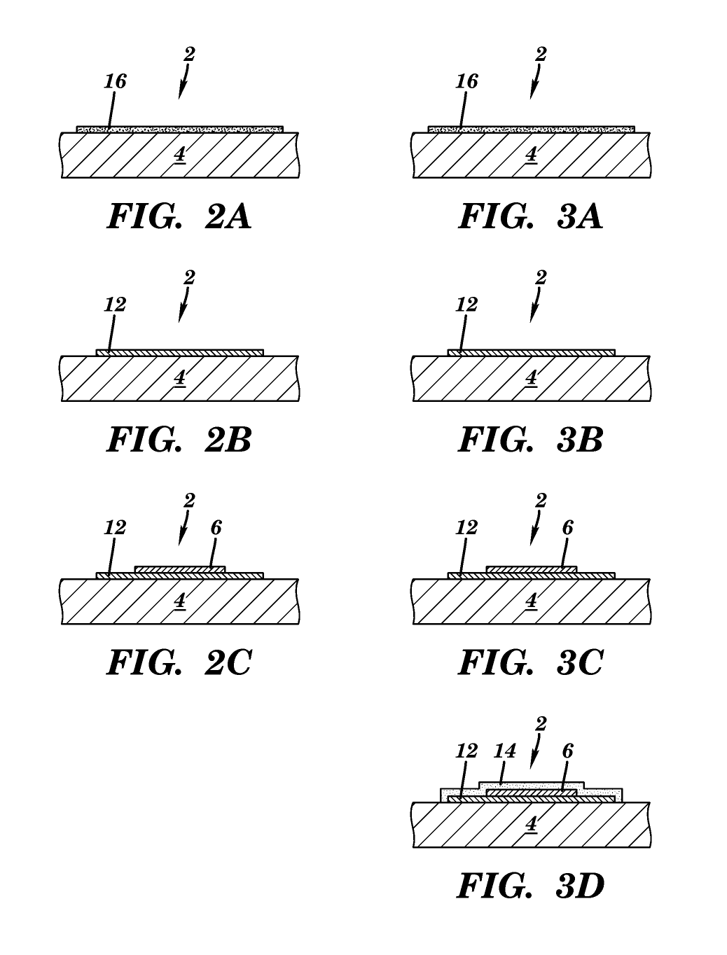

With reference now to FIGS. 2A-C, shown are horizontal, cross-sectional views of a sequence of steps, beginning with FIG. 2A, of the formation of an electrical stimulation electrode on an exposed surface of an implant where bone growth is desired according to one embodiment of the present invention. In particular, FIG. 2C is a cross-sectional view of implant 2 (FIG. 1C) with electrical stimulation electrode 6 formed on insulating substrate layer 12, which is in turn formed on surface 4.

Thus, one embodiment of a fabrication sequence for electrical stimulation electrode 6 on surface 4 of implant 2 is shown in FIGS. 2A-C. FIG. 2A depicts the application of intermediate layer 16 to surface 4. Intermediate layer 16 is an optional layer which may be used, for example, to improve adhesion between surface 4 of implant 2 and electrical stimulation electrode 6 and any insulating substrate layer 12. Note that for simplicity of presentation, intermediate layer 16 is omitted in FIG. 2B and FIG. 2C. FIG. 2B shows insulating substrate layer 12 which, as noted supra, is also optional depending upon the nature of the material of surface 4 in that particular region of exposed surface 4 of implant 2. As noted supra, the function of insulating substrate layer 12 is to provide electric isolation if implant 2 possesses an electronic or ionic conductivity such that it interferes with the proper functioning of electrical stimulation electrode 6. The presence of insulating substrate layer 12 is illustrated in FIG. 2B and FIG. 2C, because frequently surface 4 is constructed of a metal material. However, in those cases where the surface is not metal, the insulating substrate layer could still be present, but would be superfluous from an electrical isolation point of view. FIG. 2C shows that electrical stimulation electrode 6 is formed over a portion of insulating substrate layer 12.

When employed, the intermediate layer, such as intermediate layer 16, may be formed for example, from metalorganic or organometallic species, olefin, epoxy, cyanoacrylate, polyacrylate, natural rubber, polyester, polyethylene napthalate, polypropylene, polystyrene, polyvinyl fluoride ethyl-vinyl acetate, ethylene acrylic acid, acetyl polymer, poly(vinyl chloride), silicone, polyurethane, polyisoprene, styrene-butadiene, acrylonitrile-butadiene-styrene, polyethylene, polyamide, polyether-amide, polyimide, polyetherimide, polyetheretherketone, polyvinylidene chloride, polyvinylidene fluoride, polycarbonate, polysulfone, polytetrafuoroethylene, polyethylene terephthalate, polyhydroxyalkanoate, poly(p-xylylene), liquid crystal polymer, polymethylmethacrylate, polyhydroxyethylmethacrylate, polylactic acid, polyhydroxyvalerate, polyphosphazene, poly(ε-caprolactone), and mixtures or copolymers thereof.

With reference now to FIGS. 3A-D, illustrated are additional steps that may be carried out to perform the method of the present invention. In particular, FIGS. 3A-D are horizontal, cross-sectional views of implant 2 with insulating substrate layer 12, electrical stimulation electrode 6, and insulating top layer 14 formed on surface 4 (see FIG. 1C). Thus, another embodiment of a fabrication sequence for electrical stimulation electrode 6 on surface 4 of implant 2 is shown in FIGS. 3A-D. In FIG. 3A, optional intermediate layer 16 is applied to surface 4 for adhesion promotion. FIG. 3B shows the application of insulating substrate layer 12. As noted supra, insulating substrate layer 12 is not necessary if surface 4 is already electrically insulating. However, in many cases device 2 is not electrically insulating. FIG. 3C shows the application of electrical stimulation electrode 6. In FIG. 3D, insulating top layer 14 is a dielectric layer disposed over the electrical stimulation electrode 6. Insulating top layer 14 protects electrical stimulation electrode 6 from ions, moisture, and friction and provides support against stress. Insulating top layer 14 may contain additives that impart desirable properties such as radiopacity, lubricity, or release of medicaments. Any biocompatible, non-conductive, impermeable polymer or ceramic insulator which is easily applied may be used (e.g., medical grade silicones, such as those provided by NuSil® (Bakersfield, Calif.), or medical grade acrylate adhesives, such as those provided by Dymax® (Torrington, Conn.)).

Turning now to FIG. 4, illustrated is an alternative embodiment of the osseous implant of the present invention. In particular, FIG. 4 is a front view of a schematic illustration of dental implant 102 having an electrical circuit attached to implant 102 by direct writing to form a trace of conductive particles deposited on an exposed surface of the osseous implant according to one embodiment of the present invention. The electrical circuit includes insulating substrate layer 112, electrical stimulation electrode 106, lead 108, and insulating top layer 114. Electrical stimulation electrode 106 is formed on exposed surface 104 of implant 102 in an area in which bone growth is desired. Surface 104 of dental implant 102 is positioned into bone segment 120 which, in the illustration of FIG. 4 is cut away to show features of the implant, with the aid of threads 118.

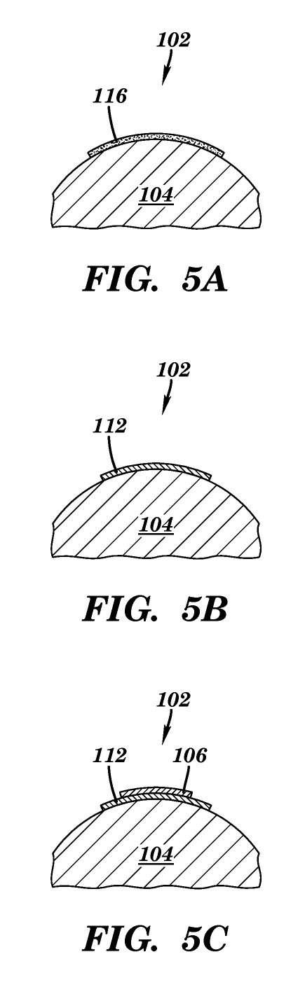

FIGS. 5A-C are horizontal, cross-sectional views showing a sequence of steps, from FIG. 5A to FIG. 5C, of the formation of electrical stimulation electrode 106 on exposed surface 104 of dental implant 102 according to one embodiment of the present invention. In FIG. 5A, optional intermediate layer 116 is applied to surface 104 for adhesion promotion. FIG. 5B shows the application of insulating substrate layer 112. As noted supra, insulating substrate layer 112 is not necessary if surface 104 is already electrically insulating. However, in many cases device 102 is not electrically insulating. FIG. 5C is a cross-sectional view of the dental implant of FIG. 4 at a region without an insulating top layer.

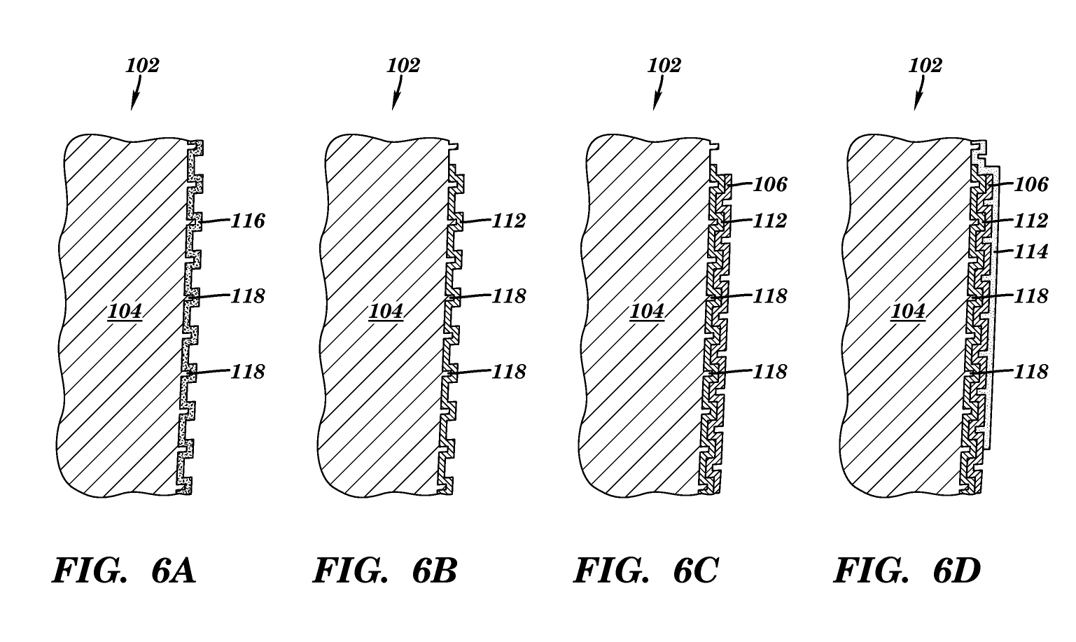

FIGS. 6A-D are vertical, cross-sectional views showing a sequence of steps, from FIG. 6A to FIG. 6D, of the formation of the electrical circuit and insulating layer of dental implant 102. The vertical cross-sectional views illustrate that the electrical circuit on surface 104 is applied, e.g., via Micropenning® along non-planar surface 104 to conform to threads 118. In an alternative embodiment, the electrical circuit is applied in a helical pattern on or between threads 118. The cross-sectional view of FIG. 6D is of the dental implant of FIG. 4 at a region with an insulating top layer. The electrical circuit includes insulating substrate layer 112, electrical stimulation electrode 106, and insulating top layer 114. In FIG. 6A, optional intermediate layer 116 is applied to surface 104 for adhesion promotion. FIG. 6B shows the application of insulating substrate layer 112. As noted supra, insulating substrate layer 112 is not necessary if surface 104 is already electrically insulating. However, in many cases device 102 is not electrically insulating. FIG. 6C shows the application of electrical stimulation electrode 106. In FIG. 6D, insulating top layer 114 is a dielectric layer disposed over the electrical stimulation electrode 106. Insulating top layer 114 protects electrical stimulation electrode 106, and may contain additives that impart desirable properties such as radiopacity, lubricity, or release of medicaments. Any biocompatible, non-conductive, impermeable polymer or ceramic insulator which is easily applied may be used (e.g., medical grade silicones, such as those provided by NuSil® (Bakersfield, Calif.), or medical grade acrylate adhesives, such as those provided by Dymax® (Torrington, Conn.)).

FIG. 7 illustrates a segment of bone scaffolding 204 designed to replace or repair bone. For example, bone scaffolding 204 may be comprised of a ceramic or polymeric material, or a combination of the two, and is often designed to be absorbed or eroded back into the body after bone has grown and interpenetrated the bone scaffolding structure. While it is conceivable to apply an osseoinductive or osteogenerative electrical stimulation electrode 206 within the porous structure of bone scaffolding 204, for simplicity, electrical stimulation electrode 206 is shown on the outside of bone scaffolding 204 in FIG. 7. In FIG. 7, an insulating top layer 214 is applied over a portion of electrical stimulation electrode 206, where stimulation may not be desired, which then leads to lead 208, which in turn is connected to DC current source 210.

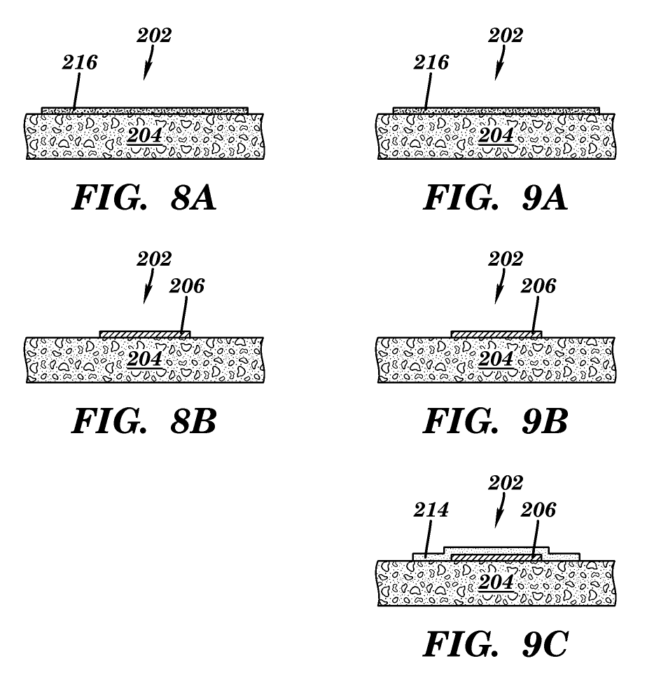

FIGS. 8A-B are horizontal, cross-sectional views showing a sequence of steps, from FIG. 8A to FIG. 8B, of electrical stimulation electrode 206 attached to bone scaffold segment 202. In FIG. 8A, optional intermediate layer 216 is applied to surface 204 to improve adhesion between surface 204 and electrical stimulation electrode 206 printed thereon. Note that for simplicity of presentation, the presence of intermediate layer 216 is omitted in FIG. 8B. FIG. 8B shows the application of electrical stimulation electrode 206. FIG. 8B is a cross-sectional view of the bone scaffold of FIG. 7 at a region without an insulating top layer.

FIGS. 9A-C are horizontal, cross-sectional views showing a sequence of steps, from FIG. 9A to FIG. 9C, of the formation of electrical stimulation electrode 206 on surface 204. In FIG. 9A, optional intermediate layer 216 is applied to surface 204 for adhesion promotion. Optional intermediate layer 216 is omitted in FIGS. 9B-C to simplify these illustrations. FIG. 9B shows the application of electrical stimulation electrode 206 onto exposed surface 204. In FIG. 9C, insulating top layer 214 is a dielectric layer disposed over electrical stimulation electrode 206. Insulating top layer 214 protects the printed electrical stimulation electrode 206 from ions, moisture, and friction and provides support against stress. Insulating top layer 214 may contain additives that impart desirable properties such as radiopacity, lubricity, or release of medicaments. Any biocompatible, non-conductive, impermeable polymer or ceramic insulator which is easily applied may be used (e.g., medical grade silicones, such as those provided by NuSil® (Bakersfield, Calif.), or medical grade acrylate adhesives, such as those provided by Dymax® (Torrington, Conn.)). If bone scaffolding 202 is intended to be resorbable, especially preferred materials would include bioresorbable polymers or ceramics. FIG. 9C is a cross-sectional view of bone scaffold segment 202 of FIG. 7 at a region with insulating layer 214.

### EXAMPLES

The following examples are provided to illustrate embodiments of the present invention but are by no means intended to limit its scope.

### Example 1

### Bioresorbable Osteogenetic Stimulation Electrode

Insulating Layer

A titanium based femoral implant was rinsed with ethanol. An electrically insulating ink was made by first dissolving polycaprolactone (Mn 70,000-90,000; Sigma-Aldrich, St. Louis, Mo.) in cyclohexanone at a concentration of 20% by weight. Hydroxyapatite (nanopowder, <200 nm particle size, Sigma-Aldrich, St. Louis, Mo.) was added to this solution to yield an ink with a weight ratio of polycaprolactone:hydroxyapatite 1:1. The hydroxyapatite was mixed into the polycaprolactone solution using a centrifugal planetary mixer (Mazerustar KK400, Kurabo Industries, Ltd., Osaka, Japan). The ink was dispensed by a syringe technique onto the femoral implant surface in a thin uniform layer to form an insulating substrate layer, and then cured at 80° C. for 30 minutes to remove the solvent.

Conductive Lead and Electrode

A conductive ink was made by first dissolving polycaprolactone (Mn 70,000-90,000; Sigma-Aldrich, St. Louis, Mo.) in cyclohexanone at a concentration of 20% by weight. Tungsten (Grade WP-100, <1 micron, Atlantic Equipment Engineers, division of Micron Metals, Bergenfield, N.J.) was added to this solution to yield an ink with a weight ratio of polycaprolactone:tungsten 1:19. The tungsten was mixed into the polycaprolactone solution using a centrifugal planetary mixer (Mazerustar KK400, Kurabo Industries, Ltd., Osaka, Japan). The ink was dispensed by a syringe technique onto the insulating substrate layer in a thin line to form an electrical stimulation electrode and then cured at 80° C. for 45 minutes to remove the solvent. The resulting line was 2 mm wide by 40 mm in length, and had a resistance of 3000 ohms.

Insulating Overcoat

The ink used in the insulating substrate layer above was syringe dispensed over half the length of the conductive trace (i.e., electrical stimulation electrode), and cured at 80° C. for 30 minutes, providing an insulating top layer over a portion of the electrical stimulation electrode while leaving the electrical stimulation electrode portion exposed.

### Example 2

### Biostable Osteogenetic Stimulation Electrode

Insulating Layer

A titanium based femoral implant was rinsed with ethanol. An electrically insulating ink, Dymax 1-20323, was dispensed by a syringe technique onto the femoral implant surface in a thin uniform layer to form an insulating substrate layer, and cured by ultraviolet radiation.

Conductive Lead and Electrode

Next a conductive ink, CMI 101-59 (Creative Materials, Inc., Ayer, Mass.), was dispensed by a syringe technique onto the insulating substrate layer in a thin line to form an electrical stimulation electrode, and then cured at 120° C. for 30 minutes to remove the solvent. The resulting line was 2 mm wide by 40 mm in length, and had a resistance of 0.7 ohms.

Insulating Overcoat

The ink used in the insulating substrate layer above was syringe dispensed over half the length of the electrical stimulation electrode, and then cured by ultraviolet radiation, to form an insulating top layer over the lead portion of the electrical stimulation electrode while leaving a portion of the electrical stimulation electrode exposed.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An osseous implant for osteogenesis promotion and maintenance, said implant having an exposed surface, the improvement comprising:
an electrical circuit attached to said osseous implant, wherein at least a portion of the electrical circuit comprises a trace of conductive particles deposited on the exposed surface of the osseous implant.

2. The osseous implant according to claim 1, wherein at least a portion of the electrical circuit is above the exposed surface of the osseous implant.

3. The osseous implant according to claim 1, wherein at least a portion of the electrical circuit is formed from a cured conductive ink composition.

4. The osseous implant according to claim 3, wherein the conductive ink composition comprises conductive particles in a liquid carrier.

5. A method of promoting and maintaining osteogenesis, said method comprising:
providing the osseous implant according to claim 1;
implanting the osseous implant into a subject; and
passing current through the electrical circuit under conditions effective to promote and maintain osteogenesis in the subject.

6. The method according to claim 5, wherein at least a portion of the electrical circuit is above the exposed surface of the osseous implant.

7. The method according to claim 5, wherein at least a portion of the electrical circuit is formed from a cured conductive ink composition.

8. The method according to claim 7, wherein the conductive ink composition comprises conductive particles in a liquid carrier.

9. A method of making an osseous implant for osteogenesis promotion and maintenance, said method comprising:
providing an osseous implant with an exposed surface;
applying a conductive ink composition comprising conductive particles in a solvent on a surface of the osseous implant to form an electrical circuit; and
curing the conductive ink composition under conditions effective to form an electrical circuit comprising a trace of conductive particles deposited on the exposed surface of the osseous implant.

10. The method according to claim 9, wherein at least a portion of the electrical circuit is above the exposed surface of the osseous implant.

11. The method according to claim 9, wherein the conductive ink composition comprises conductive particles in a liquid carrier.
